(19) United States
(12) Patent Application Publication
(10) Pub. No.: US 2014/0228801 A1
(43) Pub. Date: Aug. 14, 2014

(54) BODY CAVITY DRAINAGE DEVICE AND METHODS FOR USING THE SAME

(71) Applicant: Gary Keeling, Bridgeville, PA (US)

(72) Inventor: Gary Keeling, Bridgeville, PA (US)

(21) Appl. No.: 14/177,138

(22) Filed: Feb. 10, 2014

Related U.S. Application Data
(60) Provisional application No. 61/902,658, filed on Nov. 11, 2013.
(60) Provisional application No. 61/763,887, filed on Feb. 12, 2013.
(60) Provisional application No. 61/810,100, filed on Apr. 9, 2013.

(51) Int. Cl. A61M 25/00; A61M 27/00

(52) U.S. Cl. CPC A61M 25/0023; A61M 27/00; A61M 2210/1017; A61M 2210/1021; A61M 2205/00; A61M 2202/0468
USPC 604/500; 604/543; 604/523

(57) ABSTRACT

The present invention provides a drainage tube and method that removes fluid from a peritoneal, abdominal, or other body site. The tube has a central internal passageway for the flow of fluids. The tubing has one or more crossholes that extend through the tubing material and provide fluid communication between the passageway of the drain tube and the peritoneal, abdominal, or other body site of a subject.

### CROSS REFERENCE TO RELATED APPLICATIONS

Pursuant to 35 U.S.C. §119 (e), this application claims priority to the filing date of U.S. Provisional Patent Application Ser. No. 61/902,658 filed on Nov. 11, 2013; U.S. Provisional Application No. 61/763,887, filed on Feb. 12, 2013 and U.S. Provisional Application No. 61/810,100 filed on Apr. 9, 2013; all of which applications are incorporated herein by reference.

### INTRODUCTION

The chemotherapeutic treatment of human cancers located within the peritoneal, abdominal, or other body cavity space is a growing field of medicine. The peritoneum is the inner lining found throughout the abdominal cavity. Cancer cells can spread into the peritoneum from tumors originally located in the colon, pancreas, appendices, gastrointestinal tract, ovaries, and other organs. Typically, when cancerous cells have spread (metastasized) from one location to another, doctors will prescribe systemic chemotherapeutic compounds by injection into a patient's bloodstream. The goal of such methods is to destroy wayward cancer cells by saturating a patient's body with what amount to chemical poisons. This is effective for a number of metastatic cancers.

However, such methods are far less effective for peritoneal cancers, because the abdominal lining has significantly less blood flow than other tissues in the body, thus creating what is referred to in the art as a “peritoneal barrier.”

Accordingly, the penetration of chemotherapeutic chemicals into the peritoneal cavity is very low. Reacting to this challenge, medical researchers developed a procedure called intraperitoneal hyperthermic chemo-perfusion or “HIPEC.” HIPEC is a treatment for cancers located within the peritoneal space. It is a procedure performed in an operating room, typically immediately following tumor debulking/removal surgery. HIPEC delivers what systemic administration cannot—the direct contact of anti-cancer (chemotherapeutic) compounds to the cancerous cells found within the peritoneal area. Researchers discovered that heating the circulating chemotherapy solution further increased its' ability to kill cancer cells in the patient's abdomen. The use of HIPEC is expanding, worldwide.

Typically, in HIPEC and related procedures, chemotherapeutic agents are added to approximately four liters of a sterile solution, heated and circulated by a heat exchanger and hyperthermia pump, and circulated into, and then out of, the patient's abdomen and finally back through the same cycle (forming a circuit). This procedure typically lasts for 90 to 120 minutes. The goal of this, and related procedures, is to kill remaining cancer cells that the surgeon was not able to extract during debulking surgery.

Typically, in HIPEC, and related procedures, a solution containing one or more chemotherapeutic agents is washed into and out of the peritoneal space of a subject in need, and oftentimes the solution is heated to between 40-43 C in order to enhance the cancer-killing ability of the chemotherapeutic agents within the solution. For these procedures to effectively kill cancer cells located within the peritoneum of these subjects, the solution needs to be rapidly and effectively: transferred through the subject's peritoneum, drained out of the peritoneum, be warmed by the heating element, typically located some distance from the subject, and finally be re-introduced into the subject's peritoneum at the target temperature range. Any delays, complications, or decreases in the in-flow or out-flow of the solution in this circuit can result in variances away from the ideal temperature range of the solution. Such variations are believed to decrease the cancer-killing effectiveness of the chemotherapeutic, and/or other, agents contained in the solution. (See Abstract reproduced in Appendix A of priority U.S. Provisional Patent Application Ser. No. 61/902,658 filed on Nov. 11, 2013; the disclosure of which is herein incorporated by reference).

Tubing and catheters are typically assembled to form a circuit from the patient to the HIPEC device, typically, approximately three to five feet from the surgical table. The surgeon typically places two catheters into the patient's abdomen through one or more laparotomy incisions. These catheters, (typically called in-flow catheters), are used to pump the HIPEC solution into the patient's abdominal cavity. Catheters used to drain the solution, (typically called out-flow catheters), are typically placed in the opposite end of the abdominal cavity through one or more laparotomy incisions. The laparotomy incision(s) is typically closed with one or more sutures and the skin is typically pulled tightly around the in-flow and out-flow catheters to inhibit leakage of the solution. Typically, tubing, or similar material, is connected to the catheters in order to form a closed circuit between the subject's peritoneal space and the heating and pumping devices.

Once the tubing is connected the device, the solution can now be pumped in to, and out of, the patient, and then back to the HIPEC device. Typical flow rates within this circuit are approximately 1.5 to 2 liters per minute.

It is desirable for the fluid to flow through the heat exchanger at 1.5 to 2 liters per minute to maintain a solution temperature range within the therapeutic window (see Abstract reproduced in Appendix A of the).

One of the biggest challenges with treating cancers within the peritoneal, abdominal, or other body cavities has been that, unfortunately, out-flow catheter drains oftentimes become clogged with bowel, surgical debris, and the like. Additionally, such catheters oftentimes migrate away from their original placement locations and/or come in close contact with the abdominal wall and/or bowels. Such contact prevents the orderly flow of the HIPEC solution back out of the subject to the heating and pumping devices, thereby creating interruptions, cessations, and drops in flow rate, triggering concomitant decreases in the temperature of the chemotherapeutic, or other therapeutic, solution.

Numerous catheters or drainage tubes have been developed to aide in the removal of fluids from surgical sites, abdominal spaces, etc. In our experience, however, currently used out-flow catheters clog, or otherwise have decreases, interruptions, and the like, in their drainage functioning in approximately 15 to 20% of cases. Such loss, or inhibition, of drainage causes concomitant interruptions in the steady flow of solution within the circuit from patient to heating and pumping devices. Thus, clogging, decreases, interruptions, and the like, of out-flow catheter drainage from the subject directly leads to improper heating, and therefore, decreases in the effectiveness of the intended therapeutic treatment.

Accordingly, there remains a need for improved drainage devices and methods, particularly for new devices and methods that lessen the occurrence of drainage interruption and clogging.

### SUMMARY

The present invention pertains to devices comprising drainage tubes for draining fluid from a body cavity and the method of placing those drainage tubes within a body cavity of a subject. More particularly, the present invention relates to the physical structure of such drainage tubes and their methods of manufacture.

The present invention is directed to devices and methods pertaining to a peritoneal, abdominal, or other body cavity drainage system that demonstrates enhanced flow rate, drainage rate, and clog-free drainage time, while also increasing the ability for an operator to maintain a proper temperature range of a therapeutic solution. These advances thereby enable increases in the effectiveness of chemotherapeutic, and other therapeutic solutions, with or without concomitant hyperthermia treatment.

This invention relates generally to a method and device for improving the circulation, drainage, and effectiveness of a liquid-based solution into and out of the peritoneal space of a subject. The method of the invention uses a drain comprising one or more, optionally flexible, tubular hoses, and like components, consisting of a hose, or hoses, with a series of physical, or “cross”, holes that: aid in the drainage of fluids, decrease clogging events, enhance flow rate in the circuit from patient to heating and/or pumping elements, and improve the ability of an operator to maintain a proper temperature range of a solution, either alone, or in combination with chemotherapeutic, and/or other therapeutic, agents.

The present invention provides a drainage tube and method that removes fluid from a peritoneal, abdominal, or other body site. The tube has a central internal passageway for the flow of fluids. The tubing has one or more crossholes that extend through the tubing material and provide fluid communication between the passageway of the drain tube and the peritoneal, abdominal, or other body site of a subject.

In one embodiment of the present invention, a lasso-shaped configuration of surgical tubing (hereinafter the “Keeling Lasso” drain) comprising one or more holes (which may be machine-made or manually punched) exerts significant improvements in one or more of: drainage rates from a subject, clog-free drainage time, flow rates of therapeutic solution within the circuit from a subject to pumping and/or heating elements, and proper maintenance of a target temperature range of the therapeutic solution being administered to the subject during HIPEC, and related, procedures. The discovered methods and devices therefore result in significant improvements in the execution of HIPEC, and related, procedures. In some instances, the discovered methods and devices increase the efficacy of therapeutic chemotherapy treatments typically used during HIPEC, and related, procedures.

Accordingly, the practice of the instant invention provides new methods and devices that enhance and/or improve one or more of: drainage rates from a subject, clog-free drainage time, flow rates of therapeutic solution within the circuit from a subject to pumping and/or heating elements, and proper temperature ranges of the therapeutic solution being administered to the subject, which is useful for HIPEC and related procedures.

Accordingly, the instant invention details the development of methods and devices that can be used in HIPEC, and related therapies, to significantly enhance the procedural efficacy of such treatments and thereby enhance the efficacy of anti-cancer compositions and patient outcomes.

In contrast to currently known designs, devices, and methods for removing fluid from a peritoneal, abdominal, or other body site, the crossholes and/or shape and/or placement of the Keeling lasso results in at least one of: enhanced and/or improved drainage rates from a subject, clog-free drainage time, flow rates of therapeutic solution within the circuit from a subject to pumping and/or heating elements, and proper temperature ranges of the therapeutic solution being administered to the subject.

Aspects of the invention include, but are not limited to:

1. A method for draining fluid from a peritoneal, abdominal, or other body site comprising: contacting the site with a lasso-shaped catheter that has one or more crossholes.
2. The method of embodiment 1, wherein the fluid being drained from the peritoneal, abdominal, or other body site comprises an effective amount of a therapeutic agent or therapeutic treatment.
3. The method of embodiment 2, wherein the therapeutic agent or therapeutic treatment is chemotherapy.
4. The method of embodiment 2, wherein the therapeutic agent or therapeutic treatment is combined with hyperthermic therapy.
5. The method of embodiment 2, wherein the therapeutic agent or therapeutic treatment is chemotherapy combined with hyperthermic therapy.
6. A method for treating a mammal diagnosed with cancer comprising: administering to the mammal a Keeling lasso drain effective to serve as an improved drain mechanism during therapeutic treatment(s).
7. The method of embodiment 6, wherein the therapeutic treatment is chemotherapy and comprises the administration of an effective amount of a chemotherapeutic agent to the mammal.
8. The method of embodiment 6, wherein the therapeutic treatment is chemotherapy combined with hyperthermic therapy of the peritoneal space.
9. A method for treating a mammal diagnosed with cancer comprising: administering to the mammal a Keeling lasso drain effective to increase drainage rates from a subject.
10. A method for treating a mammal diagnosed with cancer comprising: administering to the mammal a Keeling lasso drain effective to increase clog-free drainage time during HIPEC and related procedures.
11. A method for treating a mammal diagnosed with cancer comprising: administering to the mammal a Keeling lasso drain effective to increase flow rates of a therapeutic solution within the circuit from a subject to pumping and/or heating elements, during HIPEC and related procedures.
12. A method for treating a mammal diagnosed with cancer comprising: administering to the mammal a Keeling lasso drain effective to provide proper temperature ranges of a therapeutic solution being administered to the subject during HIPEC and related procedures.
13. A method of treating a cancer comprising administering to a subject in need thereof: an effective amount of a chemotherapeutic composition, and a Keeling lasso drain device, wherein said cancer is selected from the group consisting of systemic metastases, peritoneal, colorectal, gastric, ovarian, appendiceal, pseudomyxoma, sarcoma, and peritoneal mesothelioma.
14. A method for treating the peritoneal area of a warm-blooded animal which is afflicted by cancer, the method comprising applying the compounds or salt of one or more chemotherapeutics compounds to the afflicted area, and draining such compounds away from the peritoneal area by the use of the Keeling lasso drain device.
15. A method for killing cancer cells found in the peritoneal, abdominal, or other body cavity of a cancer patient comprising administering to said cancer patient (i) an effective amount of a chemotherapeutic solution, and (ii) the Keeling lasso drain device, wherein the amount of said chemotherapeutic agent is effective to killing cancer cells which are contained in said patient, and the Keeling lasso drain device is sufficient to drain said chemotherapeutic solution from said patient such that the chemotherapeutic solution volume exchange, into and out of said cavity, is larger in comparison to when standard drainage devices are administered.
16. A method for treating a subject having a tumor located in the peritoneal, abdominal, or other body cavity, comprising administering to a subject having a tumor located in the peritoneal, abdominal, or other body cavity a therapeutically effective amount of an chemotherapeutic solution, wherein the solution is drained from the subject via the Keeling lasso drain, and wherein the subject is a mammal, and the tumor is a systemic metastases, peritoneal, colorectal, gastric, ovarian, appendiceal, pseudomyxoma, sarcoma, or peritoneal mesothelioma.
17. A device for treating a subject having a peritoneal, abdominal, or other body cavity tumor comprising administering to the subject a solution comprised of a chemotherapeutic agent sufficient to kill tumor cells; and a Keeling lasso drain.
18. An article of manufacture, comprising: a) packaging material; b) a Keeling lasso drain; and c) a label indicating that the article is for use in treating cancers found within the peritoneal, abdominal, or other body cavity.
19. An article of manufacture for treatment of cancer located in the peritoneal, abdominal, or other body cavity comprising: a Keeling lasso drain that drains an aqueous suspension, optionally containing a chemotherapeutic agent, from the peritoneal, abdominal, or other body cavity.
20. An article of manufacture for treatment of cancer located in the peritoneal, abdominal, or other body cavity comprising: a Keeling lasso drain effective to increase drainage rates from a subject.
21. An article of manufacture for treatment of cancer located in the peritoneal, abdominal, or other body cavity comprising: a Keeling lasso drain effective to increase clog-free drainage time during HIPEC and related procedures.
22. An article of manufacture for treatment of cancer located in the peritoneal, abdominal, or other body cavity comprising: a Keeling lasso drain effective to increase flow rates of a therapeutic solution within the circuit from a subject to pumping and/or heating elements, during HIPEC and related procedures.
23. An article of manufacture for treatment of cancer located in the peritoneal, abdominal, or other body cavity comprising: a Keeling lasso drain effective to provide proper temperature ranges of a therapeutic solution being administered to the subject during HIPEC and related procedures.
24. A method for draining fluid from a body cavity comprising: connecting the Keeling Lasso to other art used drainage tubes and devices.
25. A method for draining fluid from a body cavity comprising: connecting the Keeling Lasso to know art drains thus forming one continuous drainage catheter.
26. A method for draining fluid from a body cavity comprising: connecting the Keeling Lasso to know art drains thus forming one continuous drainage catheter and thus inhibiting the unwanted migration, of the now connected catheters, to below the bowel, where catheters are more likely to clog with bowel and/or surgical debris.
27. A method for draining fluid from a body cavity comprising: locating the Keeling Lasso on top of the patient's bowel with the circular part of the Lasso situated near the diaphragm, ideally over the dome of the liver.
28. A method for draining fluid from a body cavity comprising: locating the Keeling Lasso in the abdomen from above the pelvis, so the loop end of the Lasso is up against the diaphragm, ideally over the dome of the liver.
29. A method to provide a drain for peritoneal, abdominal, or other body site comprising: contacting a lasso-shaped catheter, that has one or more crossholes, with known art drains thus forming one continuing drainage catheter which can be connected to a suction unit to complete the suction drain from the body site.
30. A method to provide a drain for peritoneal, abdominal, or other body site comprising: contacting a lasso-shaped catheter to know art drains thus forming one continuing drainage catheter which can be connected to a liquid circuit from the body site to pumping and/or heating devices located outside the body site.
31. A method to provide a simplified placement process of a drain for peritoneal, abdominal, or other body site comprising: contacting a lasso-shaped catheter, that has one or more crossholes, with known art drains thus forming one continuing drainage catheter and thus inhibiting the unwanted migration, of the now connected catheters, to below the bowel.
32. A method for draining fluid from a body cavity, the method comprising:

connecting a portion, or portions, of the Keeling Lasso with smaller holes near the bowel, and a portion, or portions, of the Keeling Lasso with larger holes near the patient's diaphragm to other art used drainage tubes and devices.

33. A method to provide a drain for peritoneal, abdominal, or other body site, the method comprising: contacting a lasso-shaped catheter, that has one or more crossholes, with known art drains thus forming one continuing drainage catheter which can be connected to a suction unit to complete the suction drain from the body site.
34. A method to provide a drain for peritoneal, abdominal, or other body site, the method comprising: contacting a lasso-shaped catheter to known art drains thus forming one continuing drainage catheter which can be connected to a liquid circuit from the body site to pumping and/or heating devices located outside the body site.
35. A method to provide a drain for peritoneal, abdominal, or other body site, the method comprising: contacting a lasso-shaped catheter that has a portion, or portions, with smaller crossholes located near the bowel, and a portion, or portions, with larger crossholes located near the patient's diaphragm to known art drains thus forming one continuing drainage catheter which can be connected to a liquid circuit from the body site to pumping and/or heating devices located outside the body site.
36. A method to provide a simplified placement process of a drain for peritoneal, abdominal, or other body site, the method comprising: contacting a lasso-shaped catheter, that has one or more crossholes, with known art drains thus forming one continuing drainage catheter and thus inhibiting the unwanted migration, of the now connected catheters, to below the bowel.
37. A method to provide a drain for a laparoscopic procedure of the peritoneal, abdominal, or other body site, the method comprising: contacting one or more distal ends of a lasso-shaped catheter to one or more laparoscopic openings in a patient, wherein the catheter comprises a distal portion, or portions, without crossholes, and a middle portion, or portions, with crossholes in a manner sufficient to produce one continuing drainage catheter which can be connected to a liquid circuit from the body site to pumping and/or heating devices located outside the body site.
38. The method according to Embodiment 37, wherein the method comprises suturing shut the laparoscopic openings.
39. The method according to Embodiment 38, wherein the method comprises fluidically connecting the catheter to a Y joint.
40. A drainage device configured for us in a HIPEC procedure, the device comprising:

a catheter comprising:

a first end, a second end, and

a central portion positioned between the first and second ends, wherein the central portion comprises one or more holes.

41. The device according to Embodiment 40, wherein the first and second ends lack holes.

### BRIEF DESCRIPTION OF THE FIGURES

FIGS. 1A and 1B provide views of a prior art device and its associated shortcomings. FIG. 1A provides a depiction of PVC tube bifurcating into a “Y” shape, with typical extension of the tubing for an additional ˜10 inches after the bifurcation point. Optionally, surgeons can cut the tubing to a desired length, depending on the size of the patient. FIG. 1B shows sucker guards 14 on then end of each line. Limitations of this device configuration and method include the fact that because the sucker guards are not connected together as one continuous unit (as disclosed in the instant invention Keeling Lasso) they can drift, or migrate, down beneath the bowel, causing unwanted disruptions in circuit flow by becoming clogged with bowel, surgical debris, etc. Such device and method inefficiencies significantly restrict fluid flow through the circuit.

FIG. 2 provides a picture showing the surgical opening into the peritoneal space of a patient. Laparotomy incisions typically go from beneath the diaphragm to above the pelvis, approx. 12 inches depending on the size of the patient. In FIG. 2, the surgeon's hands are shown with a drain device that includes a “Y joint” 3 for connecting two current art suction sleeve tubes to PVC surgical tubing—in order to form the “lasso” show in FIG. 2. Also shown in FIG. 2 is PVC surgical tubing (typically ½ or ⅜ inch diameter).

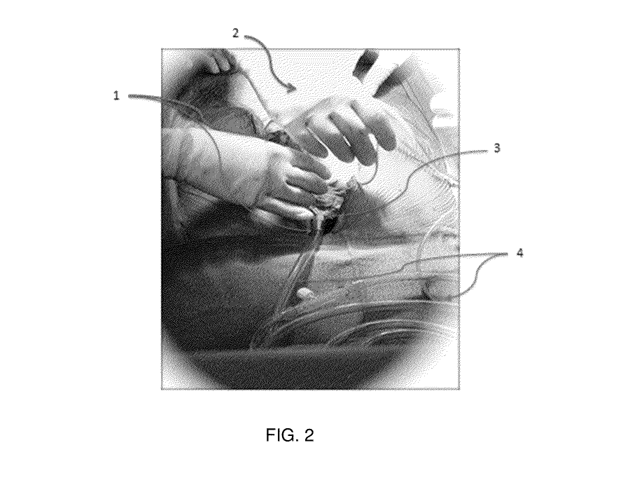

FIG. 3 provides a view of a Keeling Lasso device according to an embodiment of the invention. In FIG. 3, current art suction sleeve tubing 5 (typically ½ to ⅝ inches in diameter, tapering to ¼ inch at the tip) is operatively connect to lasso element 6, which may be a PVC surgical tubing (typically ½ or ⅜ inch diameter) that includes a plurality of machine punched holes 7 throughout the PVC tube (typically approximately 10 to 20 holes). Alternatively, holes 7 can be made with scissors, a Ronjour (a spinal surgical instrument), and the like.

FIG. 4 provides a view of a Keeling Lasso device according to another embodiment of the invention. In FIG. 4, PVC surgical tubing 8 (typically ½ or ⅜ inch diameter) is operatively connected to a “Y joint” 9 for connecting two current art suction sleeve tubes to PVC surgical tubing. Current art suction sleeve tubing components 10 (typically ½ to ⅝ inches in diameter, tapering to ¼ inch at the tip) are also present. Area of joining current art suction sleeve tubing with PVC tube with machine punched holes 11. Shown is an optional connector. An alternative embodiment is the direct connection, without a connector device, between the PVC tubing and the suction sleeve as the PVC tube can generally fits tightly over the tips of art suction sleeves. Machine punched holes 12 throughout the PVC tube (typically approximately 10 to 20 holes). Alternatively, holes can be made with scissors, a Ronjour (a spinal surgical instrument), and the like.

FIG. 5 provides a view of a Keeling Lasso device according to another embodiment of the invention. In FIG. 5, PVC surgical tubing 13 (which may be ½ or ⅜ inch diameter) is directly connected to a “Y joint” 14. Machine punched holes 15 throughout the PVC tube (for example approximately 10 to 20 holes).

FIG. 6 provides a view of a Keeling Lasso device according to an embodiment of the invention. The device is made of a PVC surgical tubing (typically ⅜ to ½ inches in diameter and 27 to 32 inches in length) that includes two distal ends of typically 9 to 10 inches in length that do not have crossholes, and a plurality of machine punched holes throughout the middle portion (typically 7 to 11 inches in length) of the PVC tube (in some instances the holes will range from 5/64 to 9/64 inches in diameter and can optionally be placed in rows, such as 2, 3 or 4 rows, with holes being approximately ⅙ of an inch apart from each other). Alternatively, holes can be made with scissors, a Ronjour (a spinal surgical instrument), and the like.

### DEFINITIONS

Unless defined otherwise, all technical and scientific terms used herein have the same meaning as is commonly understood by one of ordinary skill in the art to which this invention belongs.

As used herein, catheter, out-flow catheter, in-flow catheter, or drain, means a section of tubing, or a tube-like device, that has an internal passageway useful for transferring fluid from one location to another.

As used herein, Keeling lasso drain means a section of tubing that is optimally shaped into a lasso, or other circular, or circle-like, configuration, which contains one or more crossholes that enable fluid communication between fluid contained in a subject and the internal passageway of the section of tubing.

As used herein, chemotherapeutic agent, chemotherapeutic compound, or chemotherapeutic solution means any agent, compound, or liquid solution used in the art for the treatment of cancer. Chemotherapy can be conducted with a host of agents, and can include, among a vast array of agents: cisplatin, cisplatin-based compounds, carboplatin, mitomycin, vincristine, methotrexate, fluorouracil, calcium folinate, cytosine arabinoside, cyclophosphamide, epirubicin, etoposide, bleomycin A5, taxanes, mitoxanthrone, cylcophosphamide, topoisomerase inhibitors, angiogenesis inhibitors, cisplatin-based therapies, differentiation agents, signal transduction inhibitors, busulfan, doxorubicin rapid dissolution, etoposide, 5-fluorouracil, tamoxifen, their salts, and combinations thereof.

Additional agents, compounds, and solutions that can be used alone, or in conjunction with the chemotherapeutic agents, chemotherapeutic compounds, or chemotherapeutic solutions noted above include agents such as vitamins, anti-oxidants, natural products, minerals, etc.

As used herein, cancer refers to a general term for diseases caused by any type of tumor, and can also refer to any type of malignant growth or tumor, typically caused by abnormal and/or uncontrolled cell division.

As used herein, cancer can also refer to any malignant neoplasms that are typically characterized by the proliferation of anaplastic cells which typically displace and/or invade surrounding tissue, and that can also metastasize to other physical sites within a subject.

As used herein, an anti-cancer treatment refers to any treatment designed to treat the cancer, tumor, or neoplasm by lessening or ameliorating its symptoms. Treatments that prevent the occurrence of cancer, tumor, or neoplasm or lessen its severity are also contemplated.

As used herein, anti-cancer agent (or anti-neoplastic agent or anti-tumor agent or chemotherapy or chemotherapeutic agent) encompasses all agents and therapeutics modalities known to one of skill in the art to kill cancer cells and/or ameliorate the symptoms, in some manner, of a cancer, neoplasm, or tumor. These include any agents, used alone or in combination with other agents or compounds, can reduce, ameliorate, trigger a state of remission of symptoms or markers associated with cancers, tumors, and the like, and can be used in methods and devices provided herein.

As used herein, neoplasm refers to abnormal new growth, and thus means the same as tumor, which may be benign or malignant.

### DETAILED DESCRIPTION

The present invention provides a drainage tube and method that removes fluid from a peritoneal, abdominal, or other body site. The tube has a central internal passageway for the flow of fluids. The tubing has one or more crossholes that extend through the tubing material and provide fluid communication between the passageway of the drain tube and the peritoneal, abdominal, or other body site of a subject.

Methods and devices for peritoneal, abdominal, or other body cavity drainage are provided.

Methods and devices for peritoneal, abdominal, or other body cavity drainage that improve flow rate, drainage rate, and clog-free drainage time, while also increasing the ability for an operator to maintain a desired temperature range of a therapeutic solution are provided.

Methods and devices for treating cancers, tumors, and neoplasms using improved peritoneal, abdominal, or other body cavity drainage, either as a stand-alone treatment or in combination with one or more anti-cancer drugs or devices or other anti-neoplastic agents, treatments, or devices are provided. Also provided are kits containing the improved peritoneal, abdominal, or other body cavity drainage device.

The present invention is directed to administration of an improved peritoneal, abdominal, or other body cavity drainage device to enhance the procedural and therapeutic effectiveness of anti-cancer therapies with chemotherapeutic, or other therapeutic, agents with or without concomitant hyperthermia treatment of, and within a subject. In a specific example, administration of an improved peritoneal, abdominal, or other body cavity drainage device, in conjunction with a liquid solution of cisplatin, cisplatin-based therapies, carboplatin, mitomycin, vincristine, methotrexate, fluorouracil, calcium folinate, cytosine arabinoside, cyclophosphamide, epirubicin, etoposide, bleomycin A5, taxanes, mitoxanthrone, cylcophosphamide, topoisomerase inhibitors, angiogenesis inhibitors, cisplatin-based therapies, differentiation agents, signal transduction inhibitors, busulfan, doxorubicin rapid dissolution, etoposide, 5-fluorouracil, tamoxifen, or combinations thereof, results in a increased drainage of fluids, decreased clogging events, enhanced flow rate in the circuit from patient to heating and/or pumping elements, and increases in the ability for an operator to maintain a desired temperature range of a solution, thereby increasing the effectiveness of the therapeutic solution when compared to the effectiveness of a therapeutic solution administered with current art drains and devices.

Methods and devices of treating cancers that have metastasized to the peritoneal space, including: peritoneal, colorectal, gastric, ovarian, appendiceal, pseudomyxoma, sarcoma, peritoneal mesothelioma, and others are disclosed. The methods and devices comprise administering to a patient an improved peritoneal, abdominal, or other body cavity drainage device and a therapeutically effective amount of a chemotherapeutically active composition.

Aspects of the invention relate to methods of killing cancer cells and of creating physical conditions that enhance and improve the flow of chemotherapeutic, or other therapeutic, solutions into and out of subject's peritoneal, abdominal, or other body cavity, which involves administering to a patient an effective amount of a chemotherapeutically active composition and an improved peritoneal, abdominal, or other body cavity drainage device.

Aspects of the invention relate to methods for connecting the Keeling Lasso to known art drains, thus forming one continuing drainage catheter.

Aspects of the invention relate to methods for connecting the Keeling Lasso to know art drains thus forming one continuing drainage catheter and thus inhibiting the unwanted migration, of the now connected catheters, to below the bowel, where catheters are more likely to clog with bowel and/or surgical debris.

Aspects of the invention relate to methods for connecting the Keeling Lasso to known in the art drains, without the need for any sucker sleeves, thus forming one continuing drainage catheter and thus inhibiting the unwanted migration, of the now connected catheters, to below the bowel, where catheters are more likely to clog with bowel and/or surgical debris.

Aspects of the invention relate to methods for locating the Keeling Lasso on top of the patient's bowel with the circular part of the Lasso situated near the diaphragm, ideally over the dome of the liver.

Aspects of the invention relate to methods for locating a portion, or portions, of the Keeling lasso with smaller holes near the bowel, and a portion, or portions, of the Keeling lasso with larger holes near the diaphragm.

Aspects of the invention relate to methods for locating the Keeling Lasso in the abdomen from above the pelvis, so the loop end of the Lasso is up against the diaphragm, ideally over the dome of the liver.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is based on the discovery of an improved peritoneal, abdominal, or other body cavity drainage method and device, hereinafter referred to as the “Keeling Lasso” that can be used to aide in the drainage of fluids, decrease clogging events, enhance flow rate in the circuit from patient to heating and/or pumping elements, and increase the ability for an operator to maintain a targeted temperature range of a solution, either alone, or in combination with chemotherapeutic, or other therapeutic, agents, during HIPEC and related procedures.

Aspects of the present invention relate to a drain tube for removing fluids from within the body comprising: a tube having an internal passageway, the sidewalls of which have and at least one crosshole extending through the sidewalls, said crossholes providing for fluid communication between said internal passageway and fluid within the body. During operation, the drain is operatively coupled to a source of negative pressure, e.g., a pump, which may further be coupled to a fluid reservoir configured to receive fluid removed from the body cavity, e.g., as desired.

Aspects of the present invention relate to a drain tube, or tubes, for removing fluids from within the body comprising: a tube, or tubes, shaped in a circular, or lasso configuration, having an internal passageway, the sidewalls of which have and at least one crosshole extending through the sidewalls (e.g., 2 or more, 5 or more, 10 or more, 20 or more, such as 2 to 50, including 2 to 25 as well as 5 to 20 crossholes or analogous openings), said crossholes providing for fluid communication between said internal passageway and fluid within the body.

Aspects of the present invention relate to a device for: aiding in the drainage of fluids, decreasing clogging events, enhancing flow rate in the circuit from patient to heating and/or pumping elements, and improving the ability for an operator to maintain a targeted temperature range of a solution, either alone, or in combination with chemotherapeutic, or other therapeutic, agents, during HIPEC and related procedures.

Aspects of the present invention relate to methods and devices for enhancing the flow rate of chemotherapeutic solutions during HIPEC, and related, treatments of, and within, the peritoneal space of a subject.

Aspects of the present invention relate to methods and devices for decreasing the quantity of disruptions to the flow rate of chemotherapeutic solutions during HIPEC, and related, treatments of, and within, the peritoneal space of a subject.

Aspects of the present invention relate to methods and devices for decreasing the temporal amount of disruption to the flow rate of chemotherapeutic solutions during HIPEC, and related, treatments of, and within, the peritoneal space of a subject.

Aspects of the present invention relate to methods and devices for enhancing the flow rate in a circuit from patient to heating and/or pumping elements typically used in HIPEC, and related, procedures.

Aspects of the present invention relate to methods and devices for increasing the ability of an operator to maintain a desired temperature range of a solution, either alone, or in combination with chemotherapeutic agents.

Aspects of the present invention relate to methods and devices for enhancing the anticancer effect of chemotherapeutic compounds.

Aspects of the present invention relate to methods for connecting the Keeling lasso to other art used drainage tubes and devices.

Aspects of the present invention relate to methods for connecting the Keeling lasso to know art drains thus forming one continuous drainage catheter.

Aspects of the present invention relate to methods for connecting the Keeling lasso to know art drains thus forming one continuous drainage catheter and thereby inhibiting the unwanted migration, of the now connected catheters, to below the bowel, where catheters are more likely to clog with bowel and/or surgical debris.

Aspects of the present invention relate to methods for locating the Keeling Lasso on top of the patient's bowel with the circular part of the Keeling lasso situated near the diaphragm, ideally over the dome of the liver.

Aspects of the present invention relate to methods for locating a portion, or portions, of the Keeling Lasso with smaller holes generally near the bowel, and for locating a portion, or portions, of the Keeling Lasso with larger holes generally located near the patient's diaphragm.

Aspects of the present invention relate to methods for locating the Keeling Lasso in the abdomen from above the pelvis, so the loop end of the Lasso is up against the diaphragm, ideally over the dome of the liver.

An expanded appreciation of specific adaptations, device variations, method variants, and physical attributes will be gained upon an examination of the following detailed description of preferred embodiments.

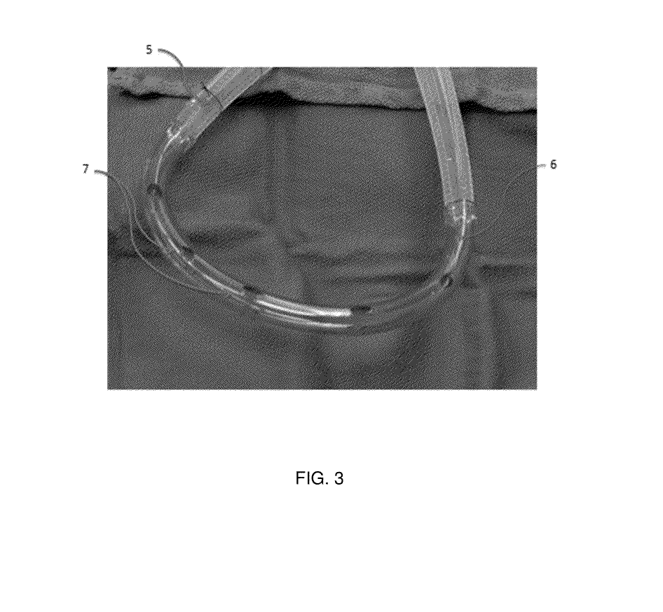

FIG. 3 provides a view of a Keeling Lasso device according to an embodiment of the invention. In FIG. 3, current art suction sleeve tubing 5 (typically ½ to ⅝ inches in diameter, tapering to ¼ inch at the tip) is operatively connect to lasso element 6, which may be a PVC surgical tubing (typically ½ or ⅜ inch diameter) that includes a plurality of machine punched holes 7 throughout the PVC tube (typically approximately 10 to 20 holes). Alternatively, holes 7 can be made with scissors, a Ronjour (a spinal surgical instrument), and the like.

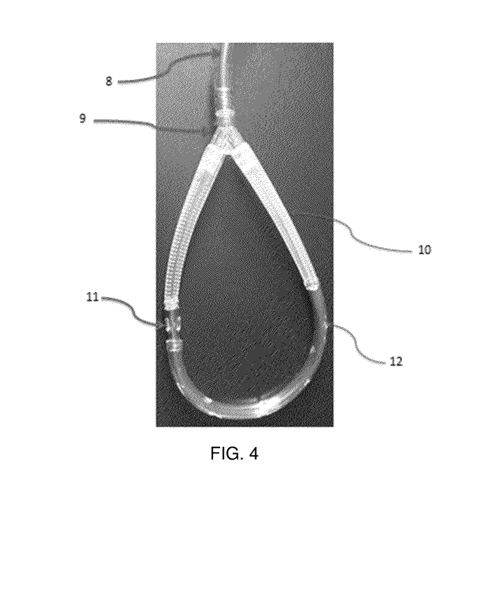

FIG. 4 provides a view of a Keeling Lasso device according to another embodiment of the invention. In FIG. 4, PVC surgical tubing 8 (typically ½ or ⅜ inch diameter) is operatively connected to a “Y joint” 9 for connecting two current art suction sleeve tubes to PVC surgical tubing. Current art suction sleeve tubing components 10 (typically ½ to ⅝ inches in diameter, tapering to ¼ inch at the tip) are also present. Area of joining current art suction sleeve tubing with PVC tube with machine punched holes 11. Shown is an optional connector. An alternative embodiment is the direct connection, without a connector device, between the PVC tubing and the suction sleeve as the PVC tube can generally fits tightly over the tips of art suction sleeves. Machine punched holes 12 throughout the PVC tube (typically approximately 10 to 20 holes). Alternatively, holes can be made with scissors, a Ronjour (a spinal surgical instrument), and the like.

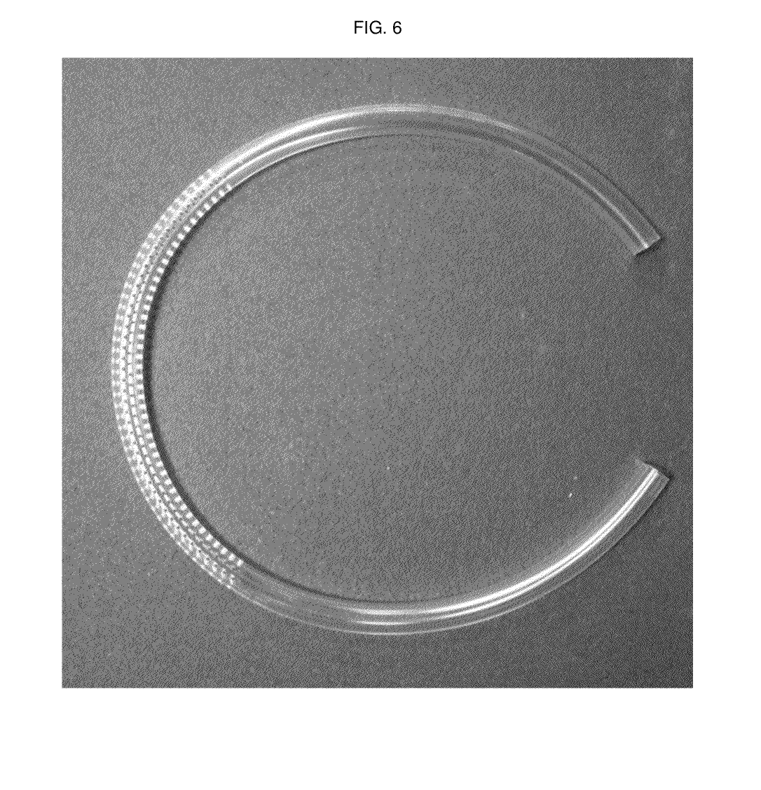

With respect to the embodiment depicted in FIG. 6, this version of the Keeling Lasso finds use in laparoscopic procedures where fluid drainage is desired. In particular, the new lasso finds use as an improved abdominal drain in cases of abdominal laparoscopic HIPEC and in other treatment regimes that do not include opening up the abdomen, or other body cavity.

In tumor burden cases where the opening up of the abdomen, or other body cavity, is not desired (e.g. for minor disease or in cases where the disease has progressed to a terminal state) surgeons typically make one or more small incisions to perform a laparoscopic version of HIPEC. Current art catheters used in such procedures suffer from similar shortcomings as do current art catheters used for non-laparoscopic (normal) HIPEC.

This instant lasso meets the particular demand of laparoscopic HIPEC by providing distal ends without crossholes that can be inserted into one or more laparoscopic incision points made in a body cavity. The distal ends may optionally be sutured to the skin of a patient to provide a closed system useful in fluid drainage from the body cavity being treated. Optionally, one distal end of the instant catheter can be pulled through and inserted into another laparoscopic opening to form a drainage system. The middle part of the instant catheter contains multiple crossholes and rests within the subject to provide a drainage system.

Also provided are kits that at least include the subject devices and which may be used according to the subject methods. The subject kits at least include a lasso device, e.g., as described above. The kits may further include one or more components to be employed in a given surgical procedure, e.g., drainage tubing, sutures, and the like. The components of the kits may be present in sterile packaging, as desired.

In certain embodiments, the kits which are disclosed herein include instructions, such as instructions for using devices. The instructions for using devices are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging etc.). In other embodiments, the instructions are present as an electronic storage data file present on a suitable computer readable storage medium, e.g., Portable Flash drive, CD-ROM, diskette, etc. The instructions may take any form, including complete instructions for how to use the device or as a website address with which instructions posted on the world wide web may be accessed.

The following examples are offered by way of illustration and not by way of limitation.

### EXPERIMENTAL

### A. Background

For the last few years we have been using prior art devices and methods during HIPEC procedures. In approximately 20% of cases, the circuit becomes clogged and/or drainage of fluids is impaired and/or flow rates within the circuit are compromised and/or interrupted and/or the operator experiences difficulty in maintaining the desired heat range of the HIPEC solution(s).

### B. Methods

The Keeling Lasso is optimally placed on top of the patient's bowel with the circular part of the lasso situated near the diaphragm, ideally over the dome of the liver. The Y is located at the pelvis end of the laparotomy, just below the skin surface with the tubing exiting the abdomen. Optionally, a portion, or portions, of the Keeling Lasso with smaller holes can be located near the bowel, and a portion, or portions, of the Keeling Lasso with larger holes can be located near the patient's diaphragm. The Keeling Lasso provides for improved perfusate fluid return from the patient when compared with current are methods and devices.

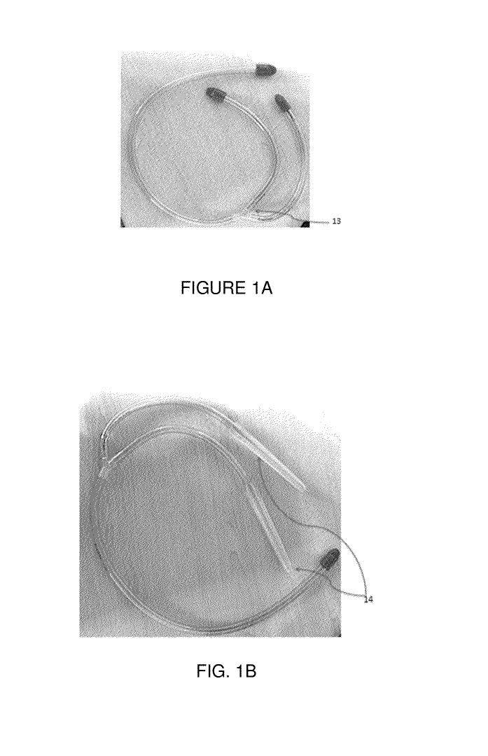

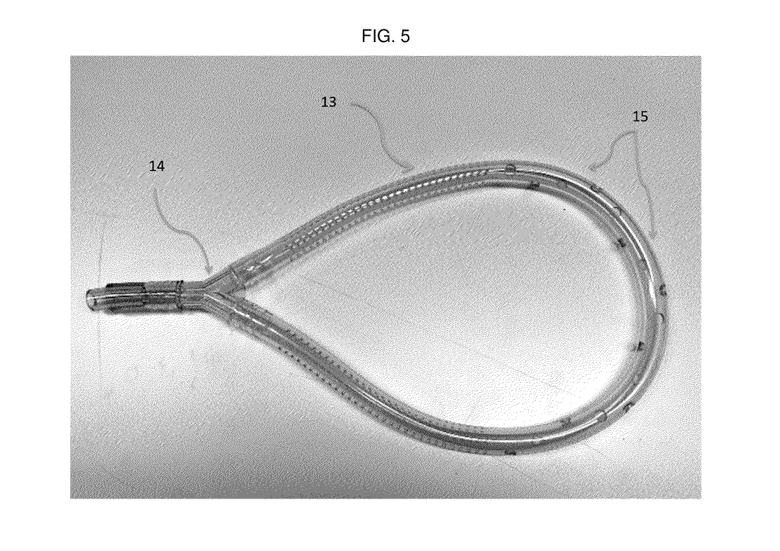

The Keeling lasso can be connected to suction sleeves, e.g., as known in the art and depicted in FIG. 1b, or the Keeling lasso can be directly connected to the drainage tubes, e.g., as depicted in FIG. 5. As show in FIG. 5, each end of the PVC tube has approximately 4-10 inches of small crossholes communicating through the PVC tube walls. This configuration alleviates the need to cut the sucker sleeves step as shown in other embodiments herein. With this embodiment, a surgeon, nurse, or technician can simply directly attach both ends of the PVC tube to the Y to form the PVC loop Keeling lasso.

### C. Results

To-date, the Keeling Lasso has been used in over 50 HIPEC cases. In 100% of these cases no clogging of the circuit has occurred, and the optimal range of solution temperatures has been reached and maintained in all cases.

Tables I to IV, attached, represent four typical flow and heat data points collected during HIPEC procedures using the present invention.

TABLE I

| | | Set | Act | Set | Heater | Valve | Patient | Patient | |
|---|---|---|---|---|---|---|---|---|---|
| Item | Time | Flow | Flow | Temp | Temp | Temp | In | Out | Timer |
| 1 | 03:13:58 | 800 | 797 | 45 | 45.1 | 43.8 | 42.5 | 36.2 | 00:00:00 |
| 2 | 03:14:58 | 1200 | 1201 | 45 | 44.7 | 43.5 | 42.6 | 38.2 | 00:00:00 |
| 3 | 03:15:58 | 1350 | 1333 | 45 | 45.8 | 43.7 | 43.4 | 39.1 | 00:00:00 |
| 4 | 03:16:58 | 1600 | 1622 | 45 | 44.8 | 42.6 | 43 | 39.4 | 00:00:00 |
| 5 | 03:17:58 | 1600 | 1599 | 45 | 45.3 | 43.4 | 43.2 | 39.7 | 00:00:00 |
| 6 | 03:18:58 | 1600 | 1598 | 45 | 45.8 | 43.6 | 43.4 | 39.9 | 00:00:00 |
| 7 | 03:19:58 | 1800 | 1804 | 45 | 45.7 | 42.9 | 43.4 | 41 | 00:00:00 |
| 8 | 03:20:58 | 1800 | 1811 | 45 | 45.4 | 43.5 | 43.3 | 41.3 | 00:00:00 |
| 9 | 03:21:58 | 1800 | 1796 | 45 | 45.9 | 43.5 | 43.4 | 41.6 | 00:00:00 |
| 10 | 03:22:59 | 1800 | 1791 | 45 | 46 | 43.6 | 43.5 | 41.8 | 00:00:00 |
| 11 | 03:23:58 | 1800 | 1794 | 45 | 46.1 | 43.7 | 43.5 | 42.2 | 00:00:30 |
| 12 | 03:24:58 | 1800 | 1795 | 44.5 | 44.8 | 42.9 | 42.7 | 41.5 | 00:01:29 |
| 13 | 03:25:58 | 1800 | 1797 | 44.5 | 45.3 | 43.1 | 43 | 41.5 | 00:02:30 |
| 14 | 03:26:58 | 1800 | 1792 | 45 | 44.8 | 42.9 | 42.6 | 41.5 | 00:03:30 |
| 15 | 03:27:59 | 1800 | 1794 | 45 | 45.3 | 43 | 42.9 | 41 | 00:04:30 |
| 16 | 03:28:58 | 1800 | 1793 | 45 | 45.4 | 43 | 42.9 | 41.5 | 00:05:30 |
| 17 | 03:29:58 | 1800 | 1797 | 45 | 45.5 | 43 | 42.9 | 41.1 | 00:06:29 |
| 18 | 03:30:58 | 1800 | 1796 | 45 | 45.6 | 43.1 | 43 | 41.5 | 00:07:30 |
| 19 | 03:31:59 | 1800 | 1791 | 45 | 45.7 | 43.2 | 43.1 | 41.9 | 00:08:30 |
| 20 | 03:32:58 | 1800 | 1799 | 45 | 45.7 | 43.2 | 43.1 | 41.9 | 00:09:29 |
| 21 | 03:33:58 | 1800 | 1791 | 45 | 45.8 | 43.2 | 43.1 | 41.1 | 00:10:30 |
| 22 | 03:34:58 | 1800 | 1795 | 45 | 45.6 | 43 | 42.9 | 40.9 | 00:11:30 |
| 23 | 03:35:58 | 1800 | 1798 | 45 | 45.7 | 43.1 | 43 | 41.3 | 00:12:29 |
| 24 | 03:36:58 | 1800 | 1808 | 45 | 45.7 | 43.2 | 43.1 | 41.2 | 00:13:30 |
| 25 | 03:37:58 | 1800 | 1804 | 45 | 45.8 | 43.3 | 43.2 | 41.2 | 00:14:30 |
| 26 | 03:38:58 | 1800 | 1793 | 45 | 45.8 | 43.4 | 43.2 | 41.4 | 00:15:29 |
| 27 | 03:39:59 | 1800 | 1808 | 45 | 45.9 | 43.4 | 43.3 | 41.4 | 00:16:30 |
| 28 | 03:40:58 | 1800 | 1791 | 45 | 46 | 43.5 | 43.3 | 41.4 | 00:17:30 |
| 29 | 03:41:58 | 1800 | 1795 | 45 | 46 | 43.5 | 43.4 | 41.4 | 00:18:29 |
| 30 | 03:42:58 | 1800 | 1799 | 45 | 46 | 43.5 | 43.4 | 41.7 | 00:19:30 |
| 31 | 03:43:58 | 1800 | 1796 | 45 | 46.1 | 43.6 | 43.5 | 42.1 | 00:20:30 |
| 32 | 03:44:58 | 1800 | 1793 | 44.5 | 46.1 | 43.5 | 43.5 | 42.5 | 00:21:29 |
| 33 | 03:45:58 | 1800 | 1803 | 44.5 | 45 | 43.2 | 43.1 | 42.1 | 00:22:30 |
| 34 | 03:46:58 | 1800 | 1794 | 44.5 | 45.4 | 42.8 | 42.9 | 41.5 | 00:23:29 |
| 35 | 03:47:59 | 1800 | 1807 | 44.5 | 45.4 | 42.9 | 42.8 | 41.3 | 00:24:30 |
| 36 | 03:48:59 | 1800 | 1797 | 44.5 | 45.6 | 43.1 | 42.9 | 41.2 | 00:25:30 |
| 37 | 03:49:58 | 1800 | 1802 | 44.5 | 45.7 | 43.1 | 43 | 41.2 | 00:26:29 |
| 38 | 03:50:58 | 1800 | 1792 | 44.5 | 45.8 | 43.2 | 43.1 | 41.1 | 00:27:30 |
| 39 | 03:51:58 | 1800 | 1800 | 45 | 45.1 | 42.9 | 42.7 | 41 | 00:28:30 |
| 40 | 03:52:58 | 1800 | 1804 | 45.5 | 45 | 43.1 | 42.7 | 41.5 | 00:29:29 |
| 41 | 03:53:58 | 1800 | 1796 | 45.5 | 45.6 | 43.3 | 43.1 | 41.4 | 00:30:30 |
| 42 | 03:54:58 | 1800 | 1808 | 45.5 | 45.8 | 43.3 | 43.2 | 41.5 | 00:31:30 |
| 43 | 03:55:58 | 1800 | 1808 | 45.5 | 45.9 | 43.4 | 43.3 | 41.6 | 00:32:29 |
| 44 | 03:56:59 | 1800 | 1803 | 45.5 | 46 | 43.5 | 43.3 | 41.4 | 00:33:30 |
| 45 | 03:57:58 | 1800 | 1809 | 45.5 | 46 | 43.6 | 43.4 | 41 | 00:34:30 |
| 46 | 03:58:58 | 1800 | 1807 | 45.5 | 46.1 | 43.6 | 43.5 | 41.1 | 00:35:29 |
| 47 | 03:59:58 | 1800 | 1800 | 45.5 | 46.2 | 43.7 | 43.6 | 41.2 | 00:36:30 |
| 48 | 04:00:58 | 1800 | 1804 | 45.5 | 46.2 | 43.7 | 43.6 | 41.5 | 00:37:30 |
| 49 | 04:01:58 | 1800 | 1802 | 45.5 | 46.2 | 43.7 | 43.6 | 41.7 | 00:38:29 |
| 50 | 04:02:58 | 1800 | 1809 | 45.5 | 46.2 | 43.7 | 43.7 | 41.8 | 00:39:30 |
| 51 | 04:03:58 | 1800 | 1790 | 45.5 | 46.3 | 43.7 | 43.7 | 42 | 00:40:30 |
| 52 | 04:04:58 | 1800 | 1793 | 45.5 | 46.3 | 43.7 | 43.7 | 42 | 00:41:29 |
| 53 | 04:05:59 | 1800 | 1806 | 45.5 | 46.3 | 43.7 | 43.7 | 42.1 | 00:42:30 |
| 54 | 04:06:58 | 1800 | 1796 | 45.5 | 46.2 | 43.6 | 43.5 | 41.3 | 00:43:30 |
| 55 | 04:07:58 | 1800 | 1810 | 45.5 | 46.1 | 43.4 | 43.4 | 41.1 | 00:44:30 |
| 56 | 04:08:58 | 1800 | 1797 | 45.5 | 45.9 | 43.3 | 43.2 | 41 | 00:45:30 |
| 57 | 04:09:58 | 1800 | 1794 | 45.5 | 46 | 43.4 | 43.3 | 41 | 00:46:29 |
| 58 | 04:10:58 | 1800 | 1801 | 45.5 | 46 | 43.5 | 43.4 | 41 | 00:47:30 |
| 59 | 04:11:58 | 1800 | 1794 | 45.5 | 46.1 | 43.5 | 43.5 | 41.1 | 00:48:30 |
| 60 | 04:12:58 | 1800 | 1794 | 45.5 | 46.2 | 43.6 | 43.5 | 41.8 | 00:49:29 |
| 61 | 04:13:59 | 1800 | 1795 | 45.5 | 46.2 | 43.5 | 43.5 | 41.2 | 00:50:30 |
| 62 | 04:14:58 | 1800 | 1795 | 45.5 | 46.1 | 43.4 | 43.4 | 41.9 | 00:51:30 |
| 63 | 04:15:58 | 1800 | 1793 | 45.5 | 46 | 43.4 | 43.3 | 41.7 | 00:52:29 |
| 64 | 04:16:58 | 1800 | 1796 | 45.5 | 46.1 | 43.5 | 43.4 | 41.5 | 00:53:30 |
| 65 | 04:17:58 | 1800 | 1794 | 45.5 | 46.1 | 43.6 | 43.5 | 41.5 | 00:54:30 |
| 66 | 04:18:58 | 1800 | 1796 | 45.5 | 46.2 | 43.6 | 43.5 | 41 | 00:55:29 |
| 67 | 04:19:58 | 1800 | 1797 | 45.5 | 46.3 | 43.7 | 43.6 | 41 | 00:56:30 |
| 68 | 04:20:58 | 1800 | 1794 | 45.5 | 46.3 | 43.7 | 43.6 | 41.1 | 00:57:30 |
| 69 | 04:21:58 | 1800 | 1796 | 45.5 | 46.3 | 43.7 | 43.6 | 41.1 | 00:58:29 |
| 70 | 04:22:59 | 1800 | 1792 | 45.5 | 46.3 | 43.7 | 43.7 | 41.1 | 00:59:30 |
| 71 | 04:23:58 | 1800 | 1795 | 45.5 | 46.3 | 43.7 | 43.6 | 41.1 | 01:00:30 |
| 72 | 04:24:58 | 1800 | 1800 | 45.5 | 46.3 | 43.7 | 43.6 | 41.1 | 01:01:29 |
| 73 | 04:25:58 | 1800 | 1799 | 45.5 | 46.1 | 43.5 | 43.4 | 41 | 01:02:30 |
| 74 | 04:26:58 | 1800 | 1798 | 45.5 | 46.1 | 43.6 | 43.5 | 41 | 01:03:30 |
| 75 | 04:27:58 | 1800 | 1811 | 45.5 | 46.2 | 43.7 | 43.6 | 41 | 01:04:30 |
| 76 | 04:28:58 | 1800 | 1794 | 45.5 | 46.2 | 43.8 | 43.6 | 41 | 01:05:30 |
| 77 | 04:29:58 | 1800 | 1792 | 45.5 | 46.3 | 43.8 | 43.7 | 41.1 | 01:06:29 |
| 78 | 04:30:59 | 1800 | 1797 | 45.5 | 46.3 | 43.9 | 43.7 | 41.2 | 01:07:30 |
| 79 | 04:31:58 | 1800 | 1794 | 45.5 | 46.4 | 43.9 | 43.8 | 41.2 | 01:08:30 |
| 80 | 04:32:58 | 1800 | 1796 | 45.5 | 46.4 | 44 | 43.8 | 41.2 | 01:09:29 |
| 81 | 04:33:58 | 1800 | 1796 | 45.5 | 46.4 | 44 | 43.8 | 41.3 | 01:10:30 |
| 82 | 04:34:58 | 1800 | 1793 | 45.5 | 46.5 | 44 | 43.9 | 41.3 | 01:11:30 |
| 83 | 04:35:58 | 1800 | 1798 | 45.5 | 46.5 | 44.1 | 43.9 | 41.4 | 01:12:29 |
| 84 | 04:36:58 | 1800 | 1793 | 45.5 | 46.6 | 44.1 | 44 | 41.4 | 01:13:30 |
| 85 | 04:37:58 | 1800 | 1796 | 45.5 | 46.6 | 44.1 | 44 | 41.5 | 01:14:30 |
| 86 | 04:38:58 | 1800 | 1802 | 45 | 46.7 | 44.2 | 44 | 41.5 | 01:15:29 |
| 87 | 04:39:59 | 1800 | 1791 | 44.5 | 44.9 | 43.5 | 43.3 | 41.3 | 01:16:30 |
| 88 | 04:40:58 | 1800 | 1797 | 45 | 45.4 | 43.1 | 43.5 | 41.2 | 01:17:30 |
| 89 | 04:41:58 | 1800 | 1809 | 45 | 45.4 | 43.6 | 43.4 | 41.2 | 01:18:29 |
| 90 | 04:42:58 | 1800 | 1796 | 45 | 45.9 | 43.7 | 43.5 | 41.2 | 01:19:30 |
| 91 | 04:43:58 | 1800 | 1809 | 45 | 46.1 | 43.7 | 43.6 | 41.2 | 01:20:30 |
| 92 | 04:44:58 | 1800 | 1809 | 45 | 46.2 | 43.8 | 43.6 | 41.3 | 01:21:29 |
| 93 | 04:45:58 | 1800 | 1805 | 45 | 46.2 | 43.8 | 43.6 | 41.4 | 01:22:30 |
| 94 | 04:46:58 | 1800 | 1807 | 45 | 46.3 | 43.9 | 43.7 | 41.4 | 01:23:30 |
| 95 | 04:47:58 | 1800 | 1807 | 45 | 46.3 | 43.9 | 43.7 | 41.4 | 01:24:30 |
| 96 | 04:48:58 | 1800 | 1806 | 45 | 46.4 | 43.9 | 43.8 | 41.4 | 01:25:30 |
| 97 | 04:49:58 | 1800 | 1796 | 45 | 46.4 | 44 | 43.8 | 41.4 | 01:26:29 |
| 98 | 04:50:59 | 1550 | 1536 | 45 | 45.3 | 43.4 | 43.2 | 41.4 | 01:27:30 |
| 99 | 04:51:58 | 1550 | 1556 | 45 | 45.9 | 43.9 | 43.7 | 41.3 | 01:28:30 |
| 100 | 04:52:58 | 1550 | 1560 | 45 | 46.2 | 44 | 43.7 | 41.3 | 01:29:29 |

TABLE II

| | | Set | Act | Set | Heater | Valve | Patient | Patient | |
|---|---|---|---|---|---|---|---|---|---|
| Item | Time | Flow | Flow | Temp | Temp | Temp | In | Out | Timer |
| 1 | 14:10:14 | 1550 | 1558 | 43 | 44.9 | 44.3 | 43.4 | 42.1 | 00:00:00 |
| 2 | 14:11:14 | 1550 | 1562 | 43 | 43.6 | 44.2 | 43.3 | 40.6 | 00:00:00 |
| 3 | 14:12:14 | 1750 | 1752 | 43 | 42.1 | 42.6 | 41.7 | 41 | 00:00:00 |
| 4 | 14:13:14 | 2100 | 2100 | 43 | 47.1 | 43.9 | 41.6 | 40.1 | 00:00:00 |
| 5 | 14:14:14 | 2100 | 2098 | 43 | 50.1 | 43.4 | 42.6 | 40.9 | 00:00:00 |
| 6 | 14:15:14 | 2100 | 2104 | 43 | 50.6 | 42.5 | 42.2 | 41.5 | 00:00:00 |
| 7 | 14:16:14 | 2100 | 2090 | 43 | 51.1 | 43.1 | 42 | 42 | 00:00:49 |
| 8 | 14:17:14 | 2100 | 2112 | 43 | 51.1 | 43.3 | 42.3 | 42.5 | 00:01:49 |
| 9 | 14:18:14 | 2100 | 2100 | 43 | 51 | 43.5 | 42.6 | 42.5 | 00:02:49 |
| 10 | 14:19:14 | 2100 | 2097 | 43 | 51 | 43.6 | 42.8 | 42.5 | 00:03:49 |
| 11 | 14:20:14 | 2100 | 2099 | 43 | 50.8 | 43.3 | 43.1 | 42.4 | 00:04:49 |
| 12 | 14:21:14 | 2100 | 2101 | 43 | 50.7 | 44 | 43 | 42.5 | 00:05:49 |
| 13 | 14:22:14 | 2100 | 2098 | 43 | 50.7 | 43.7 | 43 | 42.4 | 00:06:49 |
| 14 | 14:23:14 | 2100 | 2098 | 43 | 50.3 | 43.7 | 43.1 | 42.6 | 00:07:49 |
| 15 | 14:24:14 | 2100 | 2105 | 43 | 49.4 | 44.2 | 42.8 | 42.2 | 00:08:49 |
| 16 | 14:25:14 | 2100 | 2105 | 43 | 49.3 | 42.1 | 43.1 | 42 | 00:09:49 |
| 17 | 14:26:14 | 2100 | 2092 | 43 | 49.6 | 43.5 | 43.3 | 42 | 00:10:49 |
| 18 | 14:27:14 | 2100 | 2104 | 43 | 49.2 | 44.7 | 42.8 | 42.3 | 00:11:49 |
| 19 | 14:28:14 | 2100 | 2103 | 43 | 49.1 | 42.8 | 42.8 | 42.6 | 00:12:49 |
| 20 | 14:29:14 | 2100 | 2102 | 43 | 49.7 | 42.6 | 43.5 | 42.4 | 00:13:49 |
| 21 | 14:30:14 | 2100 | 2100 | 43 | 48.8 | 44.6 | 42.9 | 41.8 | 00:14:49 |
| 22 | 14:31:14 | 2100 | 2097 | 43 | 48.4 | 43 | 42.8 | 42.5 | 00:15:49 |
| 23 | 14:32:14 | 2100 | 2100 | 43 | 49.2 | 43.1 | 43.2 | 42.6 | 00:16:49 |
| 24 | 14:33:14 | 2100 | 2101 | 43 | 49.1 | 44 | 43 | 42.2 | 00:17:49 |
| 25 | 14:34:14 | 2100 | 2094 | 43 | 48.5 | 43.8 | 42.7 | 42 | 00:18:49 |
| 26 | 14:35:14 | 2100 | 2105 | 43 | 49 | 42.1 | 43.2 | 42.1 | 00:19:49 |
| 27 | 14:36:14 | 2100 | 2096 | 43 | 49.1 | 44.2 | 43 | 41.8 | 00:20:49 |
| 28 | 14:37:14 | 2100 | 2097 | 43 | 49 | 43.6 | 42.8 | 42.4 | 00:21:49 |
| 29 | 14:38:14 | 2100 | 2097 | 43 | 49.3 | 42.7 | 43.3 | 42.2 | 00:22:49 |
| 30 | 14:39:14 | 2100 | 2100 | 43 | 49.2 | 44 | 43.1 | 41.8 | 00:23:49 |
| 31 | 14:40:14 | 2100 | 2101 | 43 | 48.9 | 44.4 | 42.9 | 41.8 | 00:24:49 |
| 32 | 14:41:14 | 2100 | 2093 | 43 | 48.8 | 43.4 | 43 | 41.9 | 00:25:49 |
| 33 | 14:42:14 | 2100 | 2102 | 43 | 48.3 | 43.4 | 43.2 | 42.3 | 00:26:49 |
| 34 | 14:43:14 | 2100 | 2100 | 43 | 47.7 | 44.2 | 42.9 | 42 | 00:27:49 |
| 35 | 14:44:14 | 2100 | 2106 | 43 | 47.6 | 43.1 | 42.7 | 41.8 | 00:28:49 |
| 36 | 14:45:14 | 2100 | 2098 | 43 | 48.4 | 42.8 | 43.4 | 41.9 | 00:29:49 |
| 37 | 14:46:14 | 2100 | 2098 | 43 | 47.8 | 44.4 | 42.8 | 42.1 | 00:30:49 |
| 38 | 14:47:14 | 2100 | 2096 | 43 | 47.8 | 43 | 42.7 | 42.3 | 00:31:49 |
| 39 | 14:48:14 | 2100 | 2097 | 43 | 48.2 | 42.9 | 43.3 | 42.5 | 00:32:49 |
| 40 | 14:49:14 | 2100 | 2094 | 43 | 48 | 44.4 | 42.9 | 41.9 | 00:33:49 |
| 41 | 14:50:14 | 2100 | 2096 | 43 | 47.5 | 43.3 | 42.7 | 42.4 | 00:34:49 |
| 42 | 14:51:14 | 2100 | 2100 | 43 | 48.3 | 42.4 | 43.2 | 42.1 | 00:35:49 |
| 43 | 14:52:14 | 2100 | 2096 | 43 | 48.2 | 43.5 | 43.4 | 41.7 | 00:36:49 |
| 44 | 14:53:14 | 2100 | 2096 | 43 | 47.5 | 44.3 | 42.7 | 42 | 00:37:49 |
| 45 | 14:54:14 | 2100 | 2099 | 43 | 47.7 | 42.4 | 42.9 | 42.3 | 00:38:49 |
| 46 | 14:55:14 | 2100 | 2101 | 43 | 48.2 | 42.5 | 43.3 | 41.5 | 00:39:49 |
| 47 | 14:56:14 | 2100 | 2098 | 43 | 48 | 44.3 | 43.1 | 41.6 | 00:40:49 |
| 48 | 14:57:14 | 2100 | 2106 | 43 | 47.6 | 43.7 | 42.8 | 42.5 | 00:41:49 |
| 49 | 14:58:14 | 2100 | 2106 | 43 | 47.6 | 42.6 | 43.2 | 43 | 00:42:49 |
| 50 | 14:59:14 | 2100 | 2101 | 43 | 47.8 | 44 | 43.2 | 42.4 | 00:43:49 |
| 51 | 15:00:14 | 2100 | 2097 | 43 | 47.7 | 44 | 42.7 | 42.7 | 00:44:49 |
| 52 | 15:01:14 | 2100 | 2097 | 43 | 48.7 | 42.9 | 43.2 | 43.2 | 00:45:49 |
| 53 | 15:02:14 | 2100 | 2097 | 42.5 | 44.8 | 42.7 | 42.1 | 42 | 00:46:49 |
| 54 | 15:03:14 | 2100 | 2096 | 42.5 | 46.5 | 42.9 | 42.6 | 42.3 | 00:47:49 |
| 55 | 15:04:14 | 2100 | 2097 | 42.5 | 46.9 | 43.3 | 42.5 | 42.5 | 00:48:49 |
| 56 | 15:05:14 | 2100 | 2102 | 42.5 | 46.9 | 43.3 | 42.5 | 42.5 | 00:49:49 |
| 57 | 15:06:14 | 2100 | 2101 | 42.5 | 46.9 | 43.1 | 42.5 | 42.6 | 00:50:49 |
| 58 | 15:07:14 | 2100 | 2102 | 42.5 | 46.6 | 43.3 | 42.5 | 42.5 | 00:51:49 |
| 59 | 15:08:14 | 2100 | 2103 | 42.5 | 46.3 | 42.5 | 42.5 | 42.6 | 00:52:49 |
| 60 | 15:09:14 | 2100 | 2100 | 42.5 | 46.3 | 42.5 | 42.9 | 42.6 | 00:53:49 |
| 61 | 15:10:14 | 2100 | 2101 | 42.5 | 45.7 | 43.7 | 42.2 | 42.2 | 00:54:49 |
| 62 | 15:11:14 | 2100 | 2097 | 42.5 | 45.8 | 42.3 | 42.3 | 42.5 | 00:55:49 |
| 63 | 15:12:14 | 2100 | 2096 | 42.5 | 46.3 | 42.2 | 42.8 | 42.6 | 00:56:49 |
| 64 | 15:13:14 | 2100 | 2095 | 42.5 | 46.1 | 43.9 | 42.5 | 42.1 | 00:57:49 |
| 65 | 15:14:14 | 2100 | 2098 | 42.5 | 45.9 | 42.8 | 42.2 | 42.2 | 00:58:49 |
| 66 | 15:15:14 | 2100 | 2108 | 42.5 | 46.5 | 42.3 | 42.8 | 42.6 | 00:59:49 |
| 67 | 15:16:14 | 2100 | 2097 | 42.5 | 46.2 | 43.9 | 42.2 | 42.1 | 01:00:49 |
| 68 | 15:17:14 | 2100 | 2101 | 42.5 | 46.2 | 42 | 42.5 | 42.5 | 01:01:49 |
| 69 | 15:18:14 | 2100 | 2098 | 42.5 | 46.3 | 43.1 | 42.7 | 42.2 | 01:02:49 |
| 70 | 15:19:14 | 2100 | 2095 | 42.5 | 46 | 43.1 | 42.1 | 42.1 | 01:03:49 |
| 71 | 15:20:14 | 2100 | 2104 | 42.5 | 46 | 42 | 42.4 | 42.5 | 01:04:49 |
| 72 | 15:21:14 | 2100 | 2106 | 42.5 | 46.7 | 43.1 | 42.6 | 42.6 | 01:05:49 |
| 73 | 15:22:14 | 2100 | 2100 | 42.5 | 46.7 | 43.2 | 42.3 | 42.4 | 01:06:49 |
| 74 | 15:23:14 | 2100 | 2099 | 42.5 | 46.6 | 42 | 42.5 | 42.7 | 01:07:49 |
| 75 | 15:24:14 | 2100 | 2101 | 42.5 | 47 | 43 | 42.8 | 42.6 | 01:08:49 |
| 76 | 15:25:14 | 2100 | 2099 | 42.5 | 46.3 | 43.4 | 42.2 | 42.2 | 01:09:49 |
| 77 | 15:26:14 | 2100 | 2097 | 42.5 | 46.1 | 42.4 | 42.3 | 42.5 | 01:10:49 |
| 78 | 15:27:14 | 2100 | 2096 | 42.5 | 46.4 | 42.2 | 42.7 | 42.4 | 01:11:49 |
| 79 | 15:28:14 | 2100 | 2108 | 42.5 | 46.6 | 43.2 | 42.6 | 42.4 | 01:12:49 |
| 80 | 15:29:14 | 2100 | 2096 | 42.5 | 46.1 | 43.2 | 42.2 | 42.2 | 01:13:49 |
| 81 | 15:30:14 | 2100 | 2093 | 42.5 | 46.4 | 42 | 42.6 | 42.6 | 01:14:49 |
| 82 | 15:31:14 | 2100 | 2098 | 42.5 | 46.6 | 43.1 | 42.7 | 42.4 | 01:15:49 |
| 83 | 15:32:14 | 2100 | 2108 | 42.5 | 46.4 | 43.8 | 42.3 | 42.2 | 01:16:49 |
| 84 | 15:33:14 | 2100 | 2090 | 42.5 | 46.5 | 42 | 42.5 | 42.6 | 01:17:49 |
| 85 | 15:34:14 | 2100 | 2099 | 42.5 | 46.7 | 43.3 | 42.8 | 42.3 | 01:18:49 |
| 86 | 15:35:14 | 2100 | 2097 | 42.5 | 46 | 43.1 | 42.2 | 42.2 | 01:19:49 |
| 87 | 15:36:14 | 2100 | 2110 | 42.5 | 46.5 | 42 | 42.7 | 42.6 | 01:20:49 |
| 88 | 15:37:14 | 2100 | 2097 | 42.5 | 46.5 | 43.8 | 42.5 | 42.2 | 01:21:49 |
| 89 | 15:38:14 | 2100 | 2099 | 42.5 | 46.2 | 42.5 | 42.3 | 42.4 | 01:22:49 |
| 90 | 15:39:14 | 2100 | 2099 | 42.5 | 46.8 | 42.4 | 42.9 | 42.6 | 01:23:49 |
| 91 | 15:40:14 | 2100 | 2100 | 42.5 | 46.2 | 44 | 42.3 | 42.1 | 01:24:49 |
| 92 | 15:41:14 | 2100 | 2097 | 42.5 | 46 | 42.5 | 42.3 | 42.4 | 01:25:49 |
| 93 | 15:42:14 | 2100 | 2093 | 42.5 | 46.6 | 42.5 | 42.8 | 42.4 | 01:26:49 |
| 94 | 15:43:14 | 2100 | 2102 | 42.5 | 46.1 | 43.4 | 42.2 | 42.3 | 01:27:49 |
| 95 | 15:44:14 | 2100 | 2094 | 42.5 | 46.2 | 42.1 | 42.7 | 42.5 | 01:28:49 |
| 96 | 15:45:14 | 2100 | 2098 | 42.5 | 46.2 | 43.3 | 42.5 | 42.2 | 01:29:49 |

TABLE III

| | | Set | Act | Set | Heater | Valve | Patient | Patient | |
|---|---|---|---|---|---|---|---|---|---|
| Item | Time | Flow | Flow | Temp | Temp | Temp | In | Out | Timer |
| 1 | 15:28:17 | 800 | 800 | 45 | 44.9 | 45 | 44.4 | 35.1 | 00:00:00 |
| 2 | 15:29:17 | 1300 | 1302 | 45 | 44.5 | 44 | 43.8 | 39.1 | 00:00:00 |
| 3 | 15:30:17 | 1300 | 1293 | 45 | 48.2 | 44.9 | 44.7 | 39.2 | 00:00:00 |
| 4 | 15:31:17 | 1700 | 1700 | 45 | 49.3 | 44.4 | 44.2 | 41.5 | 00:00:00 |
| 5 | 15:32:17 | 1700 | 1698 | 45 | 53.3 | 44.7 | 44.7 | 40.7 | 00:00:00 |
| 6 | 15:33:17 | 1700 | 1700 | 45 | 56 | 45 | 44.8 | 40.9 | 00:00:41 |
| 7 | 15:34:17 | 950 | 1050 | 45 | 54.4 | 43.6 | 43.2 | 41.2 | 00:01:41 |
| 8 | 15:35:17 | 600 | 599 | 45 | 50.6 | 45.2 | 43.7 | 40.9 | 00:02:41 |
| 9 | 15:40:16 | 1400 | 1403 | 45 | 54.4 | 43.7 | 43.3 | 39.6 | 00:04:16 |
| 10 | 15:41:16 | 1400 | 1395 | 45 | 59.7 | 46.2 | 45.6 | 40 | 00:05:16 |
| 11 | 15:42:16 | 1650 | 1604 | 45 | 58.8 | 45 | 45 | 40.6 | 00:06:16 |
| 12 | 15:43:16 | 1650 | 1651 | 45 | 56 | 44.7 | 44.5 | 41.1 | 00:07:16 |
| 13 | 15:44:16 | 1650 | 1651 | 44.5 | 57.4 | 42.4 | 42.7 | 41.3 | 00:08:16 |
| 14 | 15:45:16 | 1650 | 1653 | 44.5 | 54.6 | 44.4 | 44.3 | 41.8 | 00:09:16 |
| 15 | 15:46:16 | 1650 | 1649 | 44.5 | 55.6 | 44.4 | 44.4 | 41.8 | 00:10:16 |
| 16 | 15:47:16 | 1650 | 1650 | 44.5 | 55.6 | 44.5 | 44.5 | 41.7 | 00:11:16 |
| 17 | 15:48:16 | 1750 | 1739 | 44.5 | 54.9 | 44.4 | 44.6 | 41.8 | 00:12:16 |
| 18 | 15:49:16 | 1750 | 1760 | 44.5 | 52.2 | 44.3 | 44.2 | 41.8 | 00:13:16 |
| 19 | 15:50:16 | 1750 | 1749 | 44.5 | 53.3 | 44.5 | 44.5 | 41.9 | 00:14:16 |
| 20 | 15:51:16 | 1750 | 1738 | 44 | 52.4 | 42.9 | 43 | 42 | 00:15:16 |
| 21 | 15:52:16 | 1750 | 1756 | 44 | 50.4 | 43.9 | 43.9 | 41.8 | 00:16:16 |
| 22 | 15:53:16 | 1750 | 1759 | 44 | 50.9 | 44 | 44 | 41.9 | 00:17:16 |
| 23 | 15:54:16 | 1750 | 1761 | 44 | 51 | 44 | 44 | 41.9 | 00:18:16 |
| 24 | 15:55:16 | 1750 | 1743 | 44 | 51 | 44 | 44 | 42 | 00:19:16 |
| 25 | 15:56:16 | 1750 | 1759 | 44 | 50.9 | 43.9 | 43.9 | 42.1 | 00:20:16 |
| 26 | 15:57:16 | 1750 | 1748 | 43.5 | 48.5 | 43.3 | 43.3 | 41.9 | 00:21:16 |
| 27 | 15:58:16 | 1750 | 1745 | 43.5 | 48.8 | 43.5 | 43.5 | 41.7 | 00:22:16 |
| 28 | 15:59:16 | 1750 | 1745 | 43.5 | 48.9 | 43.5 | 43.5 | 41.8 | 00:23:16 |
| 29 | 16:00:16 | 1750 | 1741 | 43.5 | 48.8 | 43.5 | 43.5 | 41.9 | 00:24:16 |
| 30 | 16:01:16 | 1750 | 1747 | 43.5 | 48.7 | 43.5 | 43.5 | 42 | 00:25:16 |
| 31 | 16:02:16 | 1750 | 1743 | 43.5 | 48.7 | 43.5 | 43.5 | 42 | 00:26:16 |
| 32 | 16:03:16 | 1750 | 1748 | 43.5 | 48.7 | 43.5 | 43.5 | 41.8 | 00:27:16 |
| 33 | 16:04:16 | 1750 | 1749 | 43.5 | 48.7 | 43.5 | 43.5 | 41.7 | 00:28:16 |
| 34 | 16:05:16 | 1750 | 1746 | 43.5 | 48.7 | 43.5 | 43.5 | 41.7 | 00:29:16 |
| 35 | 16:06:16 | 1750 | 1738 | 43.5 | 48.7 | 43.5 | 43.5 | 41.7 | 00:30:16 |
| 36 | 16:07:16 | 1750 | 1753 | 43.5 | 48.8 | 43.5 | 43.5 | 41.7 | 00:31:16 |
| 37 | 16:08:16 | 1750 | 1758 | 43.5 | 48.8 | 43.5 | 43.5 | 41.7 | 00:32:16 |
| 38 | 16:09:16 | 1750 | 1747 | 43.5 | 48.9 | 43.5 | 43.5 | 41.8 | 00:33:16 |
| 39 | 16:10:16 | 1750 | 1749 | 43.5 | 49 | 43.5 | 43.5 | 41.8 | 00:34:16 |
| 40 | 16:11:16 | 1750 | 1741 | 43.5 | 49 | 43.5 | 43.5 | 41.7 | 00:35:16 |
| 41 | 16:12:16 | 1750 | 1746 | 43.5 | 48.9 | 43.5 | 43.5 | 41.8 | 00:36:16 |
| 42 | 16:13:16 | 1750 | 1765 | 43.5 | 49 | 43.5 | 43.5 | 41.7 | 00:37:16 |
| 43 | 16:14:16 | 1750 | 1764 | 43.5 | 49 | 43.5 | 43.5 | 41.8 | 00:38:16 |
| 44 | 16:15:16 | 1750 | 1740 | 43.5 | 48.9 | 43.5 | 43.5 | 41.8 | 00:39:16 |
| 45 | 16:16:16 | 1750 | 1744 | 43.5 | 48.9 | 43.5 | 43.5 | 41.8 | 00:40:16 |
| 46 | 16:17:16 | 1750 | 1758 | 43.5 | 49 | 43.5 | 43.5 | 41.8 | 00:41:16 |
| 47 | 16:18:16 | 1750 | 1734 | 43.5 | 48.9 | 43.5 | 43.5 | 41.7 | 00:42:16 |
| 48 | 16:19:16 | 1750 | 1760 | 43.5 | 48.8 | 43.5 | 43.5 | 41.8 | 00:43:16 |
| 49 | 16:20:16 | 1750 | 1740 | 43.5 | 48.7 | 43.5 | 43.5 | 41.9 | 00:44:16 |
| 50 | 16:21:16 | 1750 | 1742 | 43.5 | 48.5 | 43.5 | 43.5 | 42.1 | 00:45:16 |
| 51 | 16:22:16 | 1900 | 1899 | 43.5 | 47.1 | 43.1 | 43.1 | 42 | 00:46:16 |
| 52 | 16:23:16 | 1900 | 1908 | 43.5 | 48.2 | 43.4 | 43.4 | 41.8 | 00:47:16 |
| 53 | 16:24:16 | 1900 | 1901 | 43.5 | 48.9 | 43.5 | 43.5 | 41.7 | 00:48:16 |
| 54 | 16:25:16 | 1900 | 1900 | 43.5 | 48.9 | 43.5 | 43.5 | 41.8 | 00:49:16 |
| 55 | 16:26:16 | 1900 | 1897 | 43.5 | 48.7 | 43.5 | 43.6 | 41.8 | 00:50:16 |
| 56 | 16:27:16 | 1900 | 1898 | 43.5 | 48.6 | 43.5 | 43.5 | 42 | 00:51:16 |
| 57 | 16:28:16 | 1900 | 1905 | 43.5 | 48.4 | 43.5 | 43.5 | 42.2 | 00:52:16 |
| 58 | 16:29:16 | 1900 | 1909 | 43 | 47.8 | 42.4 | 42.6 | 42 | 00:53:16 |
| 59 | 16:30:16 | 1900 | 1902 | 43 | 46 | 42.9 | 42.9 | 41.8 | 00:54:16 |
| 60 | 16:31:16 | 1900 | 1901 | 43 | 46.3 | 43 | 43 | 42 | 00:55:16 |
| 61 | 16:32:16 | 1900 | 1893 | 43 | 46.4 | 43 | 43 | 41.6 | 00:56:16 |
| 62 | 16:33:16 | 1900 | 1904 | 43 | 46.5 | 42.9 | 43 | 41.5 | 00:57:16 |
| 63 | 16:34:16 | 1900 | 1899 | 43 | 46.6 | 43 | 43 | 41.5 | 00:58:16 |
| 64 | 16:35:16 | 1900 | 1899 | 43 | 46.8 | 43 | 43 | 41.6 | 00:59:16 |
| 65 | 16:36:16 | 1900 | 1897 | 43 | 46.9 | 43 | 43 | 41.6 | 01:00:16 |
| 66 | 16:37:16 | 1900 | 1897 | 43.5 | 46.5 | 43 | 42.9 | 41.7 | 01:01:16 |
| 67 | 16:38:16 | 1900 | 1899 | 43.5 | 48.1 | 43.5 | 43.5 | 41.5 | 01:02:16 |
| 68 | 16:39:16 | 1900 | 1907 | 43.5 | 48.7 | 43.5 | 43.6 | 41.7 | 01:03:16 |
| 69 | 16:40:16 | 1900 | 1895 | 43.5 | 48.7 | 43.5 | 43.5 | 41.8 | 01:04:16 |
| 70 | 16:41:16 | 1900 | 1903 | 43.5 | 48.5 | 43.5 | 43.5 | 42 | 01:05:16 |
| 71 | 16:42:16 | 1900 | 1899 | 43.5 | 48.2 | 43.5 | 43.5 | 42.3 | 01:06:16 |
| 72 | 16:43:16 | 1900 | 1900 | 43.5 | 48 | 43.5 | 43.6 | 42.1 | 01:07:16 |
| 73 | 16:44:16 | 1900 | 1895 | 43.5 | 47.9 | 43.5 | 43.5 | 41.7 | 01:08:16 |
| 74 | 16:45:16 | 1900 | 1895 | 43.5 | 47.8 | 43.5 | 43.5 | 41.8 | 01:09:16 |
| 75 | 16:46:16 | 1900 | 1907 | 43.5 | 47.9 | 43.4 | 43.5 | 41.9 | 01:10:16 |
| 76 | 16:47:16 | 1900 | 1905 | 43.5 | 48 | 43.5 | 43.5 | 41.9 | 01:11:16 |
| 77 | 16:48:16 | 1900 | 1898 | 43.5 | 48.1 | 43.5 | 43.5 | 42 | 01:12:16 |
| 78 | 16:49:16 | 1900 | 1902 | 43.5 | 48.2 | 43.5 | 43.5 | 41.9 | 01:13:16 |
| 79 | 16:50:16 | 1900 | 1903 | 43.5 | 48.2 | 43.5 | 43.6 | 41.8 | 01:14:16 |
| 80 | 16:51:16 | 1900 | 1901 | 43.5 | 48 | 43.5 | 43.5 | 41.7 | 01:15:16 |
| 81 | 16:52:16 | 1900 | 1905 | 43.5 | 47.8 | 43.5 | 43.5 | 42.1 | 01:16:16 |
| 82 | 16:53:16 | 1900 | 1898 | 43.5 | 47.7 | 43.4 | 43.5 | 41.9 | 01:17:16 |
| 83 | 16:54:16 | 1900 | 1899 | 43.5 | 47.7 | 43.4 | 43.5 | 42.1 | 01:18:16 |
| 84 | 16:55:16 | 1900 | 1895 | 43.5 | 47.9 | 43.5 | 43.5 | 42 | 01:19:16 |
| 85 | 16:56:16 | 1900 | 1903 | 43.5 | 48 | 43.5 | 43.5 | 42 | 01:20:16 |
| 86 | 16:57:16 | 1900 | 1904 | 43 | 47.2 | 42.5 | 42.6 | 42.1 | 01:21:16 |
| 87 | 16:58:16 | 1900 | 1898 | 43 | 45.6 | 42.9 | 42.9 | 42.1 | 01:22:16 |
| 88 | 16:59:16 | 1900 | 1903 | 43 | 45.9 | 43 | 43 | 42.1 | 01:23:16 |
| 89 | 17:00:16 | 1900 | 1895 | 43 | 46.1 | 43 | 43 | 41.6 | 01:24:16 |
| 90 | 17:01:16 | 1900 | 1898 | 43.5 | 45.8 | 42.8 | 42.8 | 41.7 | 01:25:16 |

TABLE IV

| | | Set | Act | Set | Heater | Valve | Patient | Patient | |
|---|---|---|---|---|---|---|---|---|---|
| Item | Time | Flow | Flow | Temp | Temp | Temp | In | Out | Timer |
| 1 | 15:50:02 | 850 | 850 | 45 | 44.7 | 44.9 | 38.9 | 41.6 | 00:00:00 |
| 2 | 15:51:02 | 950 | 951 | 45 | 44.4 | 44.9 | 42.7 | 42.3 | 00:00:00 |
| 3 | 15:52:02 | 950 | 951 | 45 | 44.4 | 44.8 | 43.3 | 40.6 | 00:00:00 |
| 4 | 15:53:02 | 950 | 947 | 45 | 44.1 | 43.9 | 43.5 | 41 | 00:00:00 |
| 5 | 15:54:02 | 1450 | 1462 | 45 | 45 | 44 | 44 | 40.6 | 00:00:00 |
| 6 | 15:55:02 | 1800 | 1763 | 45 | 47 | 44.4 | 44.7 | 40.8 | 00:00:46 |
| 7 | 15:56:02 | 1800 | 1795 | 44 | 47.1 | 43.7 | 43.6 | 40.8 | 00:01:46 |
| 8 | 15:57:02 | 1800 | 1797 | 43.5 | 48.9 | 42 | 43.2 | 41.5 | 00:02:46 |
| 9 | 15:58:02 | 1800 | 1809 | 43.5 | 48 | 43.4 | 43.4 | 41 | 00:03:46 |
| 10 | 15:59:02 | 1800 | 1806 | 43.5 | 48.8 | 43.5 | 43.6 | 41.5 | 00:04:46 |
| 11 | 16:00:02 | 1800 | 1804 | 43.5 | 49.3 | 43.5 | 43.6 | 41.9 | 00:05:46 |
| 12 | 16:01:02 | 1800 | 1791 | 43 | 48.5 | 41.9 | 42 | 41.8 | 00:06:46 |
| 13 | 16:02:02 | 1800 | 1806 | 43 | 47.1 | 42.9 | 43 | 41.7 | 00:07:46 |
| 14 | 16:03:02 | 1800 | 1806 | 43 | 47.5 | 43 | 43.1 | 41.8 | 00:08:46 |
| 15 | 16:04:02 | 1800 | 1794 | 43 | 47.6 | 43 | 43.1 | 42 | 00:09:46 |
| 16 | 16:05:02 | 1800 | 1797 | 43 | 47.7 | 43 | 43.1 | 41.9 | 00:10:46 |
| 17 | 16:06:02 | 1800 | 1791 | 43 | 47.6 | 43 | 43.1 | 42 | 00:11:46 |
| 18 | 16:07:02 | 1800 | 1802 | 43 | 47.6 | 43 | 43.1 | 42 | 00:12:46 |
| 19 | 16:08:02 | 1800 | 1804 | 43 | 47.5 | 43 | 43.1 | 41.7 | 00:13:46 |
| 20 | 16:09:02 | 1800 | 1793 | 43 | 47.5 | 43 | 43 | 41.9 | 00:14:46 |
| 21 | 16:10:02 | 1700 | 1700 | 43 | 46.9 | 42.1 | 42 | 41.5 | 00:15:46 |
| 22 | 16:11:02 | 1700 | 1700 | 43 | 46.5 | 42.9 | 43 | 41.5 | 00:16:46 |
| 23 | 16:12:02 | 1700 | 1695 | 43 | 47 | 43 | 43 | 41.6 | 00:17:46 |
| 24 | 16:13:02 | 1700 | 1700 | 43 | 47.2 | 43 | 43 | 41.7 | 00:18:46 |
| 25 | 16:14:02 | 1700 | 1701 | 43 | 47.3 | 43 | 43 | 41.7 | 00:19:46 |
| 26 | 16:15:02 | 1700 | 1701 | 43 | 47.4 | 43 | 43 | 41.7 | 00:20:46 |
| 27 | 16:16:02 | 1700 | 1698 | 43 | 47.4 | 43 | 43 | 41.6 | 00:21:46 |
| 28 | 16:17:02 | 1700 | 1700 | 43 | 47.4 | 43 | 43 | 41.7 | 00:22:46 |
| 29 | 16:18:02 | 1700 | 1700 | 43 | 47.4 | 43 | 43.1 | 41.7 | 00:23:46 |
| 30 | 16:19:02 | 1700 | 1702 | 43 | 47.4 | 43 | 43 | 41.5 | 00:24:46 |
| 31 | 16:20:02 | 1700 | 1699 | 43 | 47.4 | 43 | 43.1 | 41.6 | 00:25:46 |
| 32 | 16:21:02 | 1700 | 1704 | 43 | 47.3 | 43 | 43.1 | 41.5 | 00:26:46 |
| 33 | 16:22:02 | 1700 | 1699 | 43 | 47.2 | 43 | 43.1 | 41.5 | 00:27:46 |
| 34 | 16:23:02 | 1700 | 1698 | 43 | 47.1 | 43 | 43.1 | 41.5 | 00:28:46 |
| 35 | 16:24:02 | 1700 | 1699 | 43 | 47.1 | 43 | 43 | 41.5 | 00:29:46 |
| 36 | 16:25:02 | 1700 | 1701 | 43 | 47 | 43 | 43.1 | 41.4 | 00:30:46 |
| 37 | 16:26:02 | 1700 | 1698 | 43 | 47 | 43 | 43.1 | 41.7 | 00:31:46 |
| 38 | 16:27:02 | 1700 | 1696 | 43 | 47 | 43 | 43 | 41.7 | 00:32:46 |
| 39 | 16:28:02 | 1700 | 1696 | 43 | 47 | 43 | 43.1 | 41.6 | 00:33:46 |
| 40 | 16:29:02 | 1700 | 1700 | 43 | 46.9 | 43 | 43 | 41.5 | 00:34:46 |
| 41 | 16:30:02 | 1700 | 1702 | 43 | 46.8 | 43 | 43.1 | 41.4 | 00:35:46 |
| 42 | 16:31:02 | 1700 | 1701 | 43 | 46.8 | 43 | 43 | 41.3 | 00:36:46 |
| 43 | 16:32:02 | 1700 | 1700 | 43 | 46.8 | 43 | 43.1 | 41.5 | 00:37:46 |
| 44 | 16:33:02 | 1700 | 1700 | 43 | 46.7 | 43 | 43 | 41.5 | 00:38:46 |
| 45 | 16:34:02 | 1700 | 1698 | 43 | 46.8 | 43 | 43.1 | 41.6 | 00:39:46 |
| 46 | 16:35:02 | 1700 | 1698 | 43 | 46.8 | 43 | 43.1 | 41.6 | 00:40:46 |
| 47 | 16:36:02 | 1700 | 1699 | 43 | 46.6 | 43 | 43 | 41.6 | 00:41:46 |
| 48 | 16:37:02 | 1700 | 1699 | 43 | 46.6 | 43 | 43 | 41.5 | 00:42:46 |
| 49 | 16:38:02 | 1700 | 1699 | 43 | 46.6 | 43 | 43 | 41.7 | 00:43:46 |
| 50 | 16:39:02 | 1700 | 1700 | 43 | 46.7 | 42.9 | 43 | 41.5 | 00:44:46 |
| 51 | 16:40:02 | 1700 | 1700 | 43 | 46.7 | 43 | 43.1 | 41.3 | 00:45:46 |
| 52 | 16:41:02 | 1700 | 1700 | 43 | 46.8 | 43 | 43.1 | 41.5 | 00:46:46 |
| 53 | 16:42:02 | 1700 | 1701 | 43 | 46.9 | 43 | 43.1 | 41.4 | 00:47:46 |
| 54 | 16:43:02 | 1700 | 1699 | 43 | 46.9 | 43 | 43 | 41.5 | 00:48:46 |
| 55 | 16:44:02 | 1700 | 1697 | 43 | 46.9 | 43 | 43.1 | 41.4 | 00:49:46 |
| 56 | 16:45:02 | 1700 | 1699 | 43 | 46.9 | 43 | 43.1 | 41.5 | 00:50:46 |
| 57 | 16:46:02 | 1700 | 1698 | 43 | 46.9 | 42.9 | 43 | 41.3 | 00:51:46 |
| 58 | 16:47:02 | 1700 | 1698 | 43 | 47 | 43 | 43.1 | 41.5 | 00:52:46 |
| 59 | 16:48:02 | 1600 | 1601 | 43 | 46 | 42.5 | 42.4 | 41.1 | 00:53:46 |
| 60 | 16:49:02 | 1700 | 1705 | 43 | 45.9 | 41.9 | 41.8 | 41.2 | 00:54:46 |
| 61 | 16:50:02 | 1700 | 1700 | 43 | 46 | 43 | 43 | 40.9 | 00:55:46 |
| 62 | 16:51:02 | 1700 | 1698 | 43 | 46.8 | 43 | 43.1 | 41.2 | 00:56:46 |
| 63 | 16:52:02 | 1700 | 1699 | 43 | 47 | 43 | 43 | 41.2 | 00:57:46 |
| 64 | 16:53:02 | 1700 | 1697 | 43 | 47 | 43 | 43.1 | 41.2 | 00:58:46 |
| 65 | 16:54:02 | 1700 | 1699 | 43 | 47 | 43 | 43.1 | 41.3 | 00:59:46 |
| 66 | 16:55:02 | 1700 | 1698 | 43 | 47 | 43 | 43.1 | 41.3 | 01:00:46 |
| 67 | 16:56:02 | 1700 | 1702 | 43 | 46.8 | 43 | 43.1 | 41 | 01:01:46 |
| 68 | 16:57:02 | 1700 | 1698 | 43 | 46.8 | 43 | 43 | 41.5 | 01:02:46 |
| 69 | 16:58:02 | 1700 | 1699 | 43 | 46.8 | 43 | 43.1 | 41.3 | 01:03:46 |
| 70 | 16:59:02 | 1700 | 1700 | 43 | 46.8 | 43 | 43.1 | 41.2 | 01:04:46 |
| 71 | 17:00:02 | 1700 | 1702 | 43 | 46.7 | 43 | 43 | 41.3 | 01:05:46 |
| 72 | 17:01:02 | 1700 | 1697 | 43 | 46.7 | 42.9 | 43 | 41.3 | 01:06:46 |
| 73 | 17:02:02 | 1700 | 1700 | 43 | 46.7 | 43 | 43.1 | 41.5 | 01:07:46 |
| 74 | 17:03:02 | 1700 | 1699 | 43 | 46.7 | 42.9 | 43 | 41 | 01:08:46 |
| 75 | 17:04:02 | 1700 | 1698 | 43 | 46.6 | 43 | 43 | 41.2 | 01:09:46 |
| 76 | 17:05:02 | 1700 | 1703 | 43 | 46.6 | 43 | 43 | 41.2 | 01:10:46 |
| 77 | 17:06:02 | 1650 | 1707 | 43 | 46.5 | 42 | 42.8 | 41.3 | 01:11:46 |
| 78 | 17:07:02 | 1600 | 1599 | 43 | 45.5 | 42.9 | 42.9 | 41.1 | 01:12:46 |
| 79 | 17:08:02 | 1600 | 1602 | 43 | 46.3 | 43 | 43.1 | 41.4 | 01:13:46 |
| 80 | 17:09:02 | 1600 | 1597 | 43 | 46.6 | 43 | 43 | 41.3 | 01:14:46 |
| 81 | 17:10:02 | 1600 | 1602 | 43 | 46.6 | 42.9 | 43 | 41 | 01:15:46 |
| 82 | 17:11:02 | 1600 | 1599 | 43 | 46.6 | 43 | 43.1 | 41.1 | 01:16:46 |
| 83 | 17:12:02 | 1600 | 1599 | 43 | 46.6 | 43 | 43.1 | 41.3 | 01:17:46 |
| 84 | 17:13:02 | 1600 | 1601 | 43 | 46.6 | 43 | 43.1 | 41 | 01:18:46 |
| 85 | 17:14:02 | 1600 | 1603 | 43 | 46.5 | 42.9 | 43 | 41.2 | 01:19:46 |
| 86 | 17:15:02 | 1600 | 1602 | 43 | 46.5 | 43 | 43.1 | 41.3 | 01:20:46 |
| 87 | 17:16:02 | 1600 | 1597 | 43 | 46.5 | 43 | 43 | 41.3 | 01:21:46 |
| 88 | 17:17:02 | 1600 | 1600 | 43 | 46.5 | 43 | 43.1 | 41.4 | 01:22:46 |
| 89 | 17:18:02 | 1600 | 1600 | 43 | 46.4 | 43 | 43.1 | 41.4 | 01:23:46 |
| 90 | 17:19:02 | 1600 | 1598 | 43 | 46.4 | 43 | 43 | 41.4 | 01:24:46 |
| 91 | 17:20:02 | 1600 | 1601 | 43 | 46.4 | 43 | 43 | 41.5 | 01:25:46 |
| 92 | 17:21:02 | 1600 | 1598 | 43 | 46.5 | 42.9 | 43 | 41.2 | 01:26:46 |
| 93 | 17:22:02 | 1600 | 1599 | 43 | 46.5 | 43 | 43 | 41.3 | 01:27:46 |
| 94 | 17:23:02 | 1600 | 1598 | 43 | 46.5 | 43 | 43.1 | 41.2 | 01:28:46 |
| 95 | 17:24:02 | 1600 | 1602 | 43 | 46.5 | 43 | 43 | 41.1 | 01:29:46 |

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A lasso-shaped catheter comprising one or more crossholes.

2. The catheter according to claim 1, wherein the catheter is configured to be placed in a body cavity.

3. The catheter according to claim 2, wherein the body cavity is selected from the group consisting of: a peritoneal body site or abdominal body site.

4. The catheter according to claim 1, wherein the catheter comprises 2 to 50 crossholes.

5. The catheter according to claim 1, wherein the catheter is configured to provide for clog-free drainage of the body cavity.

6. The catheter according to claim 1, wherein the catheter further comprises a therapeutic agent or therapeutic treatment.

7. The catheter according to claim 6, wherein the therapeutic agent or therapeutic treatment is chemotherapy.

8. The catheter according to claim 1, wherein the catheter is fluidically coupled to a drainage element.

9. A system comprising:
a lasso-shaped catheter comprising one or more crossholes; and
a pump.

10. The system according to claim 9, wherein the system further comprises a heating element.

11. The system according to claim 9, wherein the system further comprises a source of a chemotherapy.

12. A method for draining fluid from a body cavity, the method comprising:
positioning a lasso-shaped catheter one or more crossholes in the body cavity; and
draining fluid from the body cavity through the positioned catheter.

13. The method according to claim 12, wherein the body cavity comprises a peritoneal or abdominal site.

14. The method according to claim 12, wherein the fluid comprises a chemotherapy.

15. (canceled)
